(19) United States
(12) Patent Application Publication
(10) Pub. No.: US 2012/0149720 A1
(43) Pub. Date: Jun. 14, 2012

(54) VALACYCLOVIR FORMULATIONS

(75) Inventors: Tarun GOSWAMI, Delhi (IN); Shashikanth P. ISLOOR, Shimoga (IN); Vinod Kumar ARORA, Gurgaon (IN)

(73) Assignee: RANBAXY LABORATORIES LIMITED, Gurgaon (IN)

(21) Appl. No.: 13/187,798

(22) Filed: Jul. 21, 2011

(30) Foreign Application Priority Data
Jul. 30, 2010 (IN) 1802/DEL/2010

(51) Int. Cl. A61K 31/522; A61P 31/22

(52) U.S. Cl. 514/263.37

(57) ABSTRACT

The present invention relates to taste-masked pharmaceutical formulations of valacyclovir. The present invention also relates to process of preparation of valacyclovir suspensions, solutions and dry powder for reconstitution.

### FIELD OF THE INVENTION

The present invention relates to taste-masked pharmaceutical formulations of valacyclovir. The present invention also relates to a process of preparation of valacyclovir suspensions, solutions and dry powder for reconstitution.

### BACKGROUND OF THE INVENTION

Valacyclovir is the L-valyl ester of the antiviral drug aciclovir. Chemically, valacyclovir is known as L-valine, 2-[(2-amino-1,6-dihydro-6-oxo-9H-purin-9-yl)methoxy]ethyl ester. It is commercialized as the monohydrochloride salt and is structurally designated as:

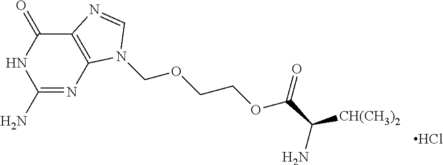

In the United States, valacyclovir is available as Valtrex® caplets for oral administration from GlaxoSmithKline. Each caplet contains valacyclovir hydrochloride equivalent to 500 mg or 1 gram valacyclovir. Valacyclovir is indicated in adult patients for the treatment and/or prophylaxis of herpes labialis, genital herpes and herpes zoster. U.S. Pat. No. 4,957,924 discloses valacyclovir and a process of preparation thereof. It also discloses pharmaceutical compositions containing valacyclovir.

Valacyclovir is also indicated for the treatment of cold sores in pediatric patients ≧12 years of age, wherein the recommended dosage is 2 grams taken twice daily. It is also indicated for the treatment of chicken pox in immunocompetent pediatric patients 2 to <18 years of age. The recommended dosage is 20 mg/kg administered three times daily for 5 days.

Valacyclovir is very difficult to formulate into a suitable dosage form. One of the most significant formulation problems encountered related is that it has an extremely bitter taste. This undesirable taste factor negatively influences patient compliance, especially with pediatric and geriatric patients.

U.S. Pat. No. 5,879,706 discloses tablet compositions of valacyclovir and colloidal silicon dioxide, along with other excipients, and a process for the preparation thereof.

The Valtrex® package insert in the United States describes an oral suspension (25 mg/mL or 50 mg/mL) formulation that may be prepared extemporaneously from the 500 mg Valtrex® (valacyclovir) tablets by crushing said tablets and processing with other excipients.

WO 01/82905 discloses pharmaceutical composition comprising carrier beads coated with one or more layers of pharmaceutically acceptable substances, wherein at least one of the said layers comprises valacyclovir, or a pharmaceutically acceptable derivative thereof, as the active ingredient.

WO 2009/049648 teaches solid dosage forms comprising valacyclovir in combination with dibasic calcium phosphate.

The most successful method for masking taste has typically involved coating valacyclovir particles with a barrier or coating layer that will not dissolve in the mouth but will readily dissolve in gastric fluids. However, such coatings may adversely impact bioavailability and/or retard release of the drug. Moreover, such processes are cumbersome and generally involve higher manufacturing costs.

The most frequently adopted techniques available in the pharmaceutical industry for masking bitter taste of drugs include taste masking with ingredients, such as flavors, and several types of sweeteners. This approach is not very reliable in masking the taste of strongly bitter tasting drugs, for example valacyclovir. Also, it is noted that a flavoring/sweetening system that works with one drug often does not apply to another drug.

Therefore, the present invention provides effective taste-masking for the bitter tasting formulations of valacyclovir. It relates to the use of a specific combination of natural sugars or sugar alcohols with artificial sweeteners for its long-lasting taste-masking effects.

### SUMMARY OF THE INVENTION

In one general aspect, the present invention provides for a taste-masked pharmaceutical formulation that includes valacyclovir and a sweetening agent, which includes a) at least one sugar or sugar alcohol; and b) at least one artificial sweetener.

Embodiments of the present invention may include one or more of the following features. For example, the artificial sweetener may be sucralose.

The sugar or sugar alcohol may be present in a concentration of about 200 to about 800 mg/mL. The artificial sweetener may be present in a concentration of about 0.1 to about 4.0 mg/mL.

Suitable sugars include one or more of glucose, fructose, invert sugar, sucrose, maltose, xylose, ribose, mannose, corn syrup solids, and mixtures thereof.

Suitable sugar alcohols include one or more of sorbitol, mannitol, xylitol and combinations thereof.

The taste-masked pharmaceutical formulation may further include one or more diluent(s), binder(s), disintegrant(s), superdisintegrant(s), flavoring agent(s), preservative(s), thickening agent(s), pH-stabilizing agent(s), lubricants(s), solvent(s), glidants(s), and moisture adsorbing agent(s) or combinations thereof. For example, the moisture adsorbing agent may be colloidal silicon dioxide.

The taste-masked pharmaceutical formulation may be in the form of a solution, suspension, or a dry powder.

In another general aspect, the present invention provides for a process for the preparation of a taste-masked pharmaceutical formulation that includes valacyclovir and sweetening agents, which includes a) at least one sugar or sugar alcohol; and b) at least one artificial sweetener. The process includes blending the valacyclovir with the sweetening agents.

### DETAILED DESCRIPTION OF THE INVENTION

The term “taste-masked pharmaceutical formulation”, as used herein, includes solid and liquid dosage forms, syrup, ready-to-use suspensions, or extemporaneously prepared liquid, syrup or suspension, such as, dry powder for reconstitution with water, liquid concentrate for dilution, or dispersible tablets or capsules. It also encompasses granules, dispersions, sublingual tablets, buccal tablets, mouth-dissolving tablets, rapidly disintegrating tablets, chewable tablets, lozenges, gums, pellets, sachets, and/or pills. Preferably, the taste-masked pharmaceutical formulation is in the form of a suspension, solution or dry powder for reconstitution.

Valacyclovir present in the pharmaceutical formulation is in “therapeutically effective amount”, i.e., in an amount that results in the alleviation and/or prophylaxis of the symptoms of the disease or condition being treated by the drug in adult, pediatric or geriatric patient population. The recommended dose of Valtrex® caplets or extemporaneously prepared suspension may be considered as the standard dose. Suitably, the formulations contain valacyclovir in an amount of about 1% to about 99%, particularly about 5% to about 25% by weight of the formulation.

The term “valacyclovir” includes without limitation, free drug and the pharmaceutically acceptable salts, esters, solvates, enantiomers, diastereomers, and polymorphs thereof, which upon administration is capable of providing valacyclovir or a physiologically and pharmaceutically active metabolite or residue thereof. In one embodiment, valacyclovir is in the form of its hydrochloride salt.

Sweetening agents in the taste-masked pharmaceutical formulation, as recited herein, include a combination of a) at least one sugar or a sugar alcohol; and b) at least one artificial sweetener.

Representative examples of sugars include glucose, fructose, invert sugar, sucrose, maltose, xylose, ribose, mannose, corn syrup solids, and mixtures thereof. The sugar alcohol includes xylitol, mannitol, sorbitol, and combinations thereof. The sugar and/or sugar alcohol may be present in amounts of from about 10% to about 99%, and in particular from about 20% to about 80%, by weight of the formulation. The concentration of the sugar and/or sugar alcohol present in the formulation may be from about 200 to about 800 mg per mL, and in particular from about 350 to about 550 mg per mL of the liquid dosage form, or when the solid dosage form is reconstituted to form a liquid dosage form.

The artificial sweeteners that may be used in the present invention include aspartame, cyclamates, saccharin, saccharin sodium, acesulfame-K, sucralose or combinations thereof The concentration of the artificial sweetener present in the formulation may be from about 0.1 to about 4.0 mg per mL, and in particular from 1.5 to about 3.0 mg per mL of the liquid dosage form, or when the solid dosage form is reconstituted to form a liquid dosage form. In one embodiment, the artificial sweetener used is sucralose. Sucralose is a highly potent artificial sweetener chemically known as 1,6-Dichloro-1,6-dideoxy-b-D-fructofuranosyl-4-chloro-4-deoxy-a-D-galactopyranoside, also known as 1′,4′,6′-trichlorogalactosucrose; and 4,1′,6′-trichloro-4,1′,6′-trideoxy-galacto-sucrose. It has a sweetening power approximately 300 to 1000 times that of sucrose and has no aftertaste. It was originally disclosed in GB 1543167. In one embodiment, sucralose, when used in combination with sugar/sugar alcohol, results in a formulation of valacyclovir which has a strong and prolonged taste-masking effect.

The taste-masked pharmaceutical formulation may further include one or more additional pharmaceutically acceptable excipient(s).

The term “pharmaceutically acceptable excipients”, as recited herein, includes pharmaceutical additives conventionally used in the preparation of solid and liquid dosage forms known in the art, such as diluent(s), binder(s), disintegrant(s), superdisintegrant(s), flavoring agent(s), preservative(s), thickening agent(s), pH-stabilizing agent(s), lubricants(s), solvent(s), glidants(s), moisture adsorbing agent(s), or combinations thereof.

Diluents may be selected from saccharides like lactose, dextrose, sucrose, fructose, maltose; sugars like mannitol, erythritol, sorbitol, xylitol and lactitol; cellulose derivatives like powdered cellulose, microcrystalline cellulose; starch and pregelatinized starch; dibasic calcium phosphate, tribasic calcium phosphate, calcium sulphate, calcium carbonate, kaolin, and the like.

Binders that may be used include starch derivatives, like corn starch and pregelatinized starch; cellulose ethers, such as carboxymethyl cellulose, methylcellulose, hydroxypropyl cellulose, hydroxypropyl methylcellulose; carboxy vinyl polymers, like carbomers; acrylates, such as Eudragits; polyvinylpyrrolidone, polyvinylpyrrolidone/vinyl acetate copolymer; xanthan gum, guar gum and other such materials routinely used in the art of pharmaceutical manufacturing.

Disintegrants and superdisintegrants may be selected from alginic acid, carboxymethylcellulose calcium, carboxymethylcellulose sodium, colloidal silicon dioxide, croscarmellose sodium, polyvinylpyrrolidone, crosslinked polyvinylpyrrolidone, guar gum, magnesium aluminium silicate, sodium starch glycolate, corn starch, potato starch, pregelatinized starch, low-substituted hydroxypropylcellulose, methylcellulose, microcrystalline cellulose, polacrilin potassium, powdered cellulose, pregelatinized starch, sodium alginate, and methacrylic acid divinylbenzene copolymer salts.

The flavoring agent(s) includes natural flavors, natural fruit flavors, artificial flavors; and artificial fruit flavors. Examples include flavors such as banana, spearmint, strawberry, and the like.

The pharmaceutical formulation may also contain a “pH-stabilizing agent”, to maintain a desired pH, such as during the process of manufacturing the formulation, and/or upon reconstitution into a solution or suspension. The desired pH may range from about 4 to about 10. The term “pH-stabilizing agent” encompasses buffers and pH-altering agents. Suitable pH-altering agents include tribasic sodium phosphate, anhydrous sodium carbonate, potassium bicarbonate, glycine, citric acid, tartaric acid and the like or mixtures thereof. The buffer system includes an aqueous mixture of an acid, such as phosphoric, succinic, tartaric, lactic, or citric acid, and a base, such as sodium hydroxide, potassium hydroxide, disodium hydrogen phosphate or sodium hydrogen carbonate.

The preservative(s) includes one or more of benzoic acid and the sodium or potassium salts thereof, sorbic acid and the sodium or potassium salts thereof, benzyl alcohol, or other preservatives well known in the art of pharmaceutical manufacturing.

Suitable thickening agents are used to increase the viscosity of the pharmaceutical liquid dosage forms. Any suitable thickening agent may be used. In practice the thickening agent is selected from the group of methyl cellulose, hydroxyethyl cellulose, hydroxypropyl cellulose, hydroxypropylmethyl cellulose, hydroxy ethyl propyl cellulose, starches (such as maize or corn starch, potato starch, rice starch, tapioca starch, and wheat starch), carboxyvinyl polymers (carbomers such as Carbopol®), carboxymethyl cellulose and salts thereof, microcrystalline cellulose and arabic gum, guar gum, and xanthan gum, and mixtures thereof.

Lubricants include magnesium stearate, calcium stearate, zinc stearate, sodium stearyl fumarate, powdered stearic acid, magnesium oleate, calcium palmitate, potassium laureate, sodium suberate, vegetable oil, mineral oil, and the like. Glidants may be selected from talc, colloidal silicon dioxide, corn starch, and the like.

Suitable solvents which may be used include water, ethanol, methanol, isopropyl alcohol, methylene chloride, acetone, and the like.

The moisture adsorbing agent includes colloidal silicon dioxide, anhydrous calcium phosphate, starch, magnesium carbonate, bentonite, kaolin, magnesium silicate, magnesium oxide and silicon dioxide, and the like.

The taste-masked pharmaceutical formulation may be prepared using conventional methods known in the art, namely blending, mixing, and granulation techniques. The formulation so formed may be administered directly or incorporated into/processed to pharmaceutical compositions for oral administration. The formulation may be introduced into a single dose or multi dose container, as a dry powder for constitution with an ingestible liquid such as water before use for administration as a suspension/solution. Typically, the dry powder after constitution in a suitable liquid will provide a liquid suspension/solution containing about 10 mg to about 250 mg valacyclovir per mL of liquid suspension/solution.

The taste-masked pharmaceutical composition may further include a moisture adsorbing agent. For example, colloidal silicon dioxide may be used.

From the above it is apparent that various modifications and combinations of the formulations detailed in the text may be made without departing from the spirit and scope of the invention. The invention as described herein may be illustrated by the following examples but is not to be construed to be limiting by them.

### EXAMPLES 1-5

| | Quantity per mL after reconstitution (in mg) | | | | |
|---|---|---|---|---|---|
| | Exam- | Exam- | Exam- | Exam- | Exam- |
| Ingredient | ple 1 | ple 2 | ple 3 | ple 4 | ple 5 |
| Valacyclovir Hydro- | 50.0 | 50.0 | 50.0 | 50.0 | 50.0 |
| chloride eq. to base | | | | | |
| Xylitol | 550.0 | 550.0 | — | — | 500.0 |
| Sucrose | — | — | 550.0 | — | — |
| Mannitol | — | — | — | 250.0 | — |
| Sucralose | 2.0 | 2.0 | 2.0 | 2.0 | 2.0 |
| Colloidal Silicon Dioxide | — | — | — | — | 6.0 |
| Sodium Benzoate | — | — | — | — | 2.0 |
| Strawberry TSL Flavor | 1.0 | 1.0 | 1.0 | 1.0 | 1.0 |
| Water | — | q.s. | q.s. | q.s. | — |

### Procedure:

### Example 1

All ingredients were weighed. Xylitol, valacyclovir hydrochloride, strawberry TSL flavor and sucralose were passed through a 30 mesh sieve and charged to a double cone blender and blended for at least 30 minutes. The requisite quantity of powder was filled in HDPE bottles to be reconstituted with water on administration.

### Example 2

All ingredients were weighed. Xylitol was added and dissolved in a stainless steel container containing purified water equivalent to 90% v/v of batch size, followed by stirring at high speed using a mechanical stirrer for a period of at least 1 hour to get a clear solution at a temperature NMT 40° C. Valacyclovir hydrochloride, strawberry TSL flavor and sucralose were added and dissolved in the xylitol solution so obtained. The solution so formed was continuously stirred for at least 1 hour to get a clear solution. The batch volume was made up using purified water. The resulting solution was filled in HDPE bottles, and sealed with child resistant caps.

### Example 3

All ingredients were weighed. Sucrose was added and dissolved in a stainless steel container containing purified water equivalent to 90% v/v of batch size, followed by stirring at high speed using a mechanical stirrer for a period of at least 1 hour to get a clear solution at a temperature NMT 40° C. Valacyclovir hydrochloride, strawberry TSL flavor and sucralose were added and dissolved in the solution of sucrose so obtained, with continuous stirring for at least 1 hour to get a clear solution. The batch volume was made up with purified water. The resulting solution was filled in HDPE bottles, and sealed with child resistant caps.

### Example 4

All ingredients were weighed. Mannitol was added and dissolved in a stainless steel container containing purified water equivalent to 90% v/v of batch size, followed by stirring at high speed using a mechanical stirrer for a period of at least 1 hour to get a clear solution at a temperature NMT 40° C. Valacyclovir hydrochloride, strawberry TSL flavor and sucralose were added and dissolved in the solution of sucrose so obtained, with continuous stirring for at least 1 hour to get a clear solution. The batch volume was made up with purified water. The resulting solution was filled in HDPE bottles, and sealed with child resistant caps.

### Example 5

All ingredients were weighed. Colloidal silicon dioxide and sodium benzoate were passed through a 44 mesh sieve and mixed with valacyclovir hydrochloride, xylitol, strawberry TSL flavor and sucralose. The material so obtained was passed through a 30 mesh sieve, and blended for at least 30 minutes in a double cone blender. The requisite quantity of powder was filled in HDPE bottles to be reconstituted with water on administration.

We claim:

1. A taste-masked pharmaceutical formulation comprising valacyclovir and sweetening agents, wherein the sweetening agents comprise of a) at least one sugar or sugar alcohol; and b) at least one artificial sweetener.

2. The taste-masked pharmaceutical formulation according to claim 1, wherein the artificial sweetener comprises sucralose.

3. The taste-masked pharmaceutical formulation according to claim 1, wherein the sugar or sugar alcohol is present in a concentration of about 200 to about 800 mg/mL.

4. The taste-masked pharmaceutical formulation according to claim 1, wherein the artificial sweetener is present in a concentration of about 0.1 to about 4.0 mg/mL.

5. The taste-masked pharmaceutical formulation according to claim 1, wherein the sugar comprises glucose, fructose, invert sugar, sucrose, maltose, xylose, ribose, mannose, corn syrup solids, and mixtures thereof.

6. The taste-masked pharmaceutical formulation according to claim 1, wherein the sugar alcohol comprises sorbitol, mannitol, xylitol, and combinations thereof.

7. The taste-masked pharmaceutical formulation according to claim 1, further comprising one or more diluent(s), binder(s), disintegrant(s), superdisintegrant(s), flavoring agent(s), preservative(s), thickening agent(s), pH-stabilizing agent(s), lubricants(s), solvent(s), glidants(s), and moisture adsorbing agent(s) or combinations thereof.

8. The taste-masked pharmaceutical formulation according to claim 7, wherein the moisture adsorbing agent comprises colloidal silicon dioxide.

9. The taste-masked pharmaceutical formulation according to claim 1, wherein the formulation is a solution.

10. The taste-masked pharmaceutical formulation according to claim 1, wherein the formulation is a suspension.

11. The taste-masked pharmaceutical formulation according to claim 1, wherein the formulation is a dry powder.

12. A process for the preparation of a taste-masked pharmaceutical formulation comprising valacyclovir and sweetening agents, wherein the sweeting agents comprising of a) at least one sugar or sugar alcohol; and b) at least one artificial sweetener, the process comprising blending the valacyclovir with the sweetening agents.
